(19) United States
(12) Patent Application Publication
(10) Pub. No.: US 2009/0264820 A1
(43) Pub. Date: Oct. 22, 2009

(54) METHOD AND APPARATUS FOR IMPLANTING AN ENDOLUMINAL PROSTHESIS SUCH AS A PROSTHETIC VALVE

(75) Inventor: Robert T. V. Kung, Andover, MA (US)

Correspondence Address: FOLEY & LARDNER LLP, 111 HUNTINGTON AVENUE, 26TH FLOOR, BOSTON, MA, 02199-7610, US

(73) Assignee: ABIOMED, INC., Danvers, MA (US)

(21) Appl. No.: 12/104,037

(22) Filed: Apr. 16, 2008

(51) Int. Cl. A61M 25/10

(52) U.S. Cl. 604/99.04

(57) ABSTRACT

Balloon catheters, and medical devices including balloon catheters, are provided that comprise a balloon assembly disposed at the distal end of an elongated tubular member. The balloon, when in the inflated state, includes a body fluid lumen that extends longitudinally through the balloon and is open at the proximal end of the balloon and the distal end of the balloon. In some aspects, the outer surface of the balloon is contiguous from the proximal end to the distal end of the balloon when the balloon is inflated. In other aspects, an endoluminal prosthesis, for example, a stent that can optionally include a prosthetic valve, surrounds the balloon assembly and is adjacent to the outer surface of the balloon. In still other aspects, endoluminal prosthesis implant devices are provided that are suitable for implanting an endoluminal prosthesis in a body vessel containing a flowing fluid such as a vessel in the heart, e.g., the left or right ventricle inflow or outflow tracks. The endoluminal prosthesis implant devices include a balloon assembly and an endoluminal prosthesis surrounding the balloon assembly. The balloon assembly is configured to permit a fluid to pass from the first end of the balloon to the second end of the balloon when the balloon is in an inflated state. In some embodiments of all of these aspects, the balloon is configured to prevent a fluid from passing from the second end of the balloon to the first end of the balloon when the balloon is in an inflated state. In some embodiments, for example, the body fluid lumen includes a body fluid lumen valve moveable between a first position and a second position. The body fluid lumen valve occludes the body fluid lumen to a greater extent in the first position than in the second position. In some embodiments, the body fluid lumen valve substantially prevents a fluid from flowing through the body fluid lumen in a first direction. Certain aspects and/or embodiments of the balloon catheters, and medical devices including balloon catheters, may allow for the deployment of an endoluminal prosthetic valve (e.g., a heart valve), while the native valve can continue to function uninterrupted under its native load and flow.

### BACKGROUND

There are numerous diseases and genetic conditions that can affect the proper function of valves in the body. For example, disease can cause stenosis of a heart valve, where the valve becomes hardened and cannot open properly, thus restricting the amount of blood that can flow through the valve. Other disease can cause incompetence of a heart valve, where the valve cannot close properly, allowing blood to flow back through the valve. At times, damaged valves must be replaced to permit the patient to lead a more active life.

### SUMMARY OF THE INVENTION

Balloon catheters have been developed that can be used to implant endoluminal prostheses (e.g., stents, graft, stent grafts, prosthetic valves and the like) in passages in the body in which it is desirable to maintain flow of a body fluid (e.g., blood) through the treatment site in a physiologic direction during the course of treatment. The balloon catheters includes a body fluid lumen through which a body fluid that ordinarily moves through the lumen being treated can pass while the balloon of the balloon catheter is inflated. The body fluid lumen can include a valve, to maintain bodily fluid flow in a single direction while the fluid is flowing through the body fluid lumen. The valve opens and closes with pressure and/or flow changes.

In a first aspect, catheters are provided that comprise an elongated tubular member having a proximal end and a distal end. A balloon assembly is disposed at the distal end of the elongated tubular member. The balloon assembly includes a balloon that has an inflated state and an uninflated state, and is typically capable of being repeatedly switched between the inflated and uninflated states. The balloon has a proximal end and a distal end. The balloon, when in the inflated state, includes a body fluid lumen that extends longitudinally through the balloon and is open at the proximal end of the balloon and the distal end of the balloon. The balloon further includes an inner surface that forms the walls or sides of the body fluid lumen and an outer surface, formed of the sides of the balloon facing away from the body fluid lumen. When the balloon is in the inflated state, the outer surface of the balloon is contiguous from the proximal end to the distal end of the balloon The elongated tubular member further includes a balloon inflation lumen in fluid communication with the balloon.

In another aspect, medical devices are provided that include an inflatable balloon assembly having a balloon with a distal end, a proximal end, and an outer surface. The balloon has an inflated state and an uninflated state, and is typically capable of being repeatedly switched between the inflated and uninflated states. The balloon, when in the inflated state, includes a body fluid lumen that extends longitudinally through the balloon and is open at the proximal end of the balloon and the distal end of the balloon. The balloon further includes an inner surface that forms the walls or sides of the body fluid lumen and an outer surface, formed of the sides of the balloon facing away from the body fluid lumen. An endoluminal prosthesis, such as, for example, a stent or graft, including, for example, a stent or graft having a prosthetic valve thereupon, surrounds the balloon assembly and is adjacent to the outer surface of the balloon.

In yet another aspect, endoluminal prosthesis implant devices are provided that are suitable for implanting an endoluminal prosthesis in a body vessel containing a flowing fluid such as, for example, an artery or vein or a vessel in the heart, e.g., the left or right ventricle inflow or outflow tracks. The endoluminal prosthesis implant devices comprise an inflatable balloon assembly that includes a balloon having an inflated state and an uninflated state and having a first end, a second end, and an outer surface. An endoluminal prosthesis surrounds the balloon assembly and is located adjacent the outer surface of the balloon. The balloon assembly is configured to permit a fluid (e.g., the body fluid in the body vessel) to pass from the first end of the balloon to the second end of the balloon when the balloon is in an inflated state, for example, when the balloon is inflated in a body vessel such that the outer surface of the balloon forms a seal against the walls of the body vessel. In some embodiments, the balloon is configured to prevent a fluid from passing from the second end of the balloon to the first end of the balloon when the balloon is in an inflated state.

In certain embodiments of any of the above aspects, the body fluid lumen includes a body fluid lumen valve, for example, a valve comprising a leaflet or leaflets, moveable between a first position and a second position. The body fluid lumen valve occludes the body fluid lumen to a greater extent in the first position than in the second position. In certain embodiments, the body fluid lumen valve substantially prevents a fluid from flowing through the body fluid lumen in a first direction.

In some embodiments having an endoluminal prosthesis, the endoluminal prosthesis is crimped to the outer surface of the balloon while the balloon is in an uninflated state. In some embodiments, the endoluminal prosthesis includes a stent. In some embodiments, the endoluminal prosthesis includes a prosthetic valve, e.g., a prosthetic aortic or mitral valve.

### BRIEF DESCRIPTION OF THE FIGURES

The accompanying drawings, which are incorporated into and constitute a part of this specification, illustrate several aspects and embodiments of the invention and together with a description of certain embodiments, serve to explain the principles of the balloon catheters disclosed herein. A brief description of the drawings is as follows:

FIG. 1 is a perspective view of an embodiment of a balloon catheter.

FIG. 2 is an exploded view in perspective of a distal portion of an embodiment of a balloon catheter.

FIG. 3 is a cross-sectional view of the distal portion of the balloon catheter illustrated in FIG. 2, wherein the balloon is in an inflated state.

FIG. 4 is a cross-sectional view of the distal portion of the balloon catheter illustrated in FIG. 2, wherein the balloon is in an uninflated state.

FIG. 5 is an exploded view in perspective of a distal portion of an embodiment of a balloon catheter.

FIG. 6 is a cross-sectional view of the distal portion of the balloon catheter illustrated in FIG. 5, wherein the balloon is in an inflated state.

FIG. 7 is a top view of an embodiment of a body fluid lumen valve.

FIGS. 8a-8c are perspective views illustrating the implantation of a prosthetic valve in a body lumen using an embodiment of a balloon catheter.

FIG. 9 is a cross-sectional view of an embodiment of a balloon catheter.

FIG. 10 is a cross-sectional view of an embodiment of a balloon catheter for maintaining balloon annular lumen shape.

FIGS. 11a-c are cross-sectional views of an embodiment of a medical device including a balloon catheter and a balloon removal configuration.

### DETAILED DESCRIPTION

The embodiments described below are not intended to be exhaustive or to limit the invention to the precise forms disclosed in the following detailed description. Rather, these embodiments are chosen and described to illustrate certain principles and practices of the present invention.

There are many circumstances that require implantation of an endoluminal prosthesis, such as, for example, a stent or a prosthetic valve. When implanting a prosthetic valve, it may be desirable that the prosthetic valve (or some portion of the implant) be located over the native valve so that the native valve is sufficiently (e.g., fully) contained so that the native valve does not unduly impede fluid flow in the body lumen once the prosthetic valve has been implanted. An implantation process may include, for example, positioning a balloon catheter in an appropriate position, inflating the balloon catheter to expand an implant into the appropriate implanted configuration, deflating the balloon catheter and then removing it.

For example, these catheters may be configured to be used to implant a replacement heart valve, such as, for example, an aortic valve, a mitral valve, a tricuspid valve, or a pulmonic valve. Prosthetic heart valves, such as, for example, valves that reside on stents, can be implanted using a balloon to expand the stent and implant it in the valve lumen such that the stent compresses the native valve against the valve lumen and the prosthetic valve on the stent opens to function in place of the native valve.

After locating the balloon and prosthetic valve at the desired location, however, fluid pressure (e.g., from the contraction of the left ventricle) can build up behind a typical balloon as it is inflated. This pressure can cause the balloon and the prosthesis to shift, for example, in the direction of fluid flow, and can cause the prosthesis to be mislocated. When implanting a prosthetic valve in place of a cardiac valve, such as the aortic valve, fluid pressure can also have negative implications for the left ventricle, which may also be subject to the pressure build-up. Also, while the typical balloon is inflated, fluid flow though the body lumen may be impeded, which can be undesirable.

Balloon catheters and methods of implanting endoluminal prostheses using balloon catheters have been developed that can, in certain embodiments, address or assist in addressing one or more of these problems. The balloon catheters include a balloon having a body fluid lumen extending through the balloon and open at either end to the body lumen being treated. The body fluid lumen allows body fluid to pass from the upstream end of the balloon to the downstream end of the balloon. The body fluid lumen includes a valve that can function in place of a native valve while the balloon is inflated, reducing or preventing retrograde flow of the body fluid. The valve in the body fluid lumen can open and close with pressure and/or flow changes.

According to one embodiment of the present invention, balloon catheters may be used, for example, to implant an endoluminal prosthesis (e.g., a replacement valve) to a treatment site that contains a valve, where it is desirable to maintain at least some fluid flow through the treatment site in a physiological direction during the course of treatment. In addition, the catheters may be configured to maintain better hemodynamics at the treatment site by reducing disruption of fluid flow patterns through the biological passage.

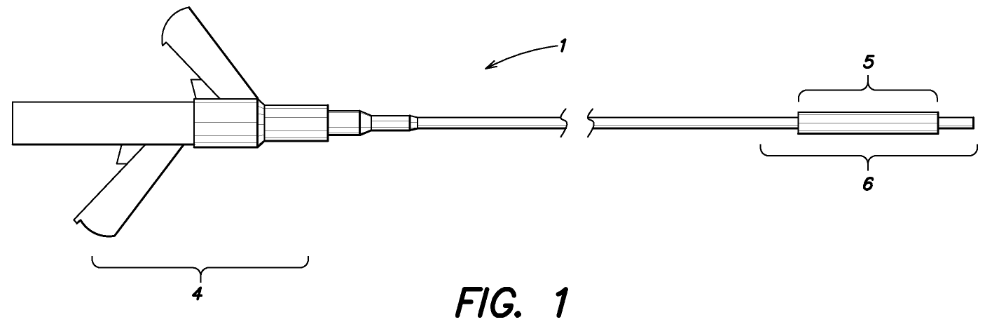

In certain embodiments, as illustrated, for example, in FIG. 1, a catheter 1 includes a proximal end 4 and distal end 6. The catheter 1 is designed such that the distal end 6 is to be inserted into a patient to effect treatment at a treatment site, while the proximal end 4 remains outside of the patient. At the proximal end 4, controls are located to allow control of the functionalities located at the distal end. The distal end 6 of the catheter includes an inflatable balloon assembly 5 for treatment of a valve, such as, for example, deployment of a prosthetic valve.

Referring to FIGS. 2-4, the catheter 1 includes a guide wire lumen 10, through which a guide wire 12 can pass. The catheter 1 includes a balloon inflation lumen 14 that is in fluid communication with an inflatable balloon 5 located at the distal end 6 of the catheter 1. In this embodiment, the balloon inflation lumen is in fluid communication with the balloon 5 via a pair of openings 16. In other embodiments, different numbers of openings can exist between the balloon inflation lumen and the balloon. For example, in some embodiments it may be advantageous to have multiple openings (e.g., three, four, five, six or more openings) along the length of the balloon, to permit the balloon to be inflated more evenly. In other embodiments, it may be advantageous to have only a single opening between the balloon inflation lumen and the balloon, to permit the balloon to be inflated in a particular direction. For example, where a stent is to be implanted over a native valve, it may be advantageous to inflate the balloon such that the balloon inflates from the upstream end (where upstream is in the context of the direction of flow of fluid in the body lumen) such that the native valve is pushed against the walls of the body lumen in the direction in which it naturally opens. The opening in such a case would be located at the upstream end of the balloon.

The balloon 5 has an outer surface 40. A stent 20 having a prosthetic valve thereupon surrounds the balloon 5, for example, by being crimped around the outer surface 40 of the balloon 5. The stent can be crimped around the balloon such that the prosthetic valve is contained between the stent body and the outer surface of the balloon, so as to present as small a profile as possible. Suitable prosthetic valves include those disclosed in U.S. Pat. Nos. 6,945,957, 5,928,281, and 4,888,009.

In use, the catheter is inserted into the body and placed at a desired location, for example, at the aortic valve. In some embodiments, the standard Seldinger technique may be used for introduction of the balloon catheter to the body. In some embodiments, after puncture of the femoral artery, the guide wire 12 is inserted into the artery and moved through the artery until it extends past the treatment site using known techniques. The catheter 1 may then be slid over the guide wire 12 until the balloon 5 of the catheter 1 is in place at the treatment site. During insertion of the catheter, in at least one embodiment, the balloon 5 is completely uninflated. After insertion, the guide wire 12 may be withdrawn.

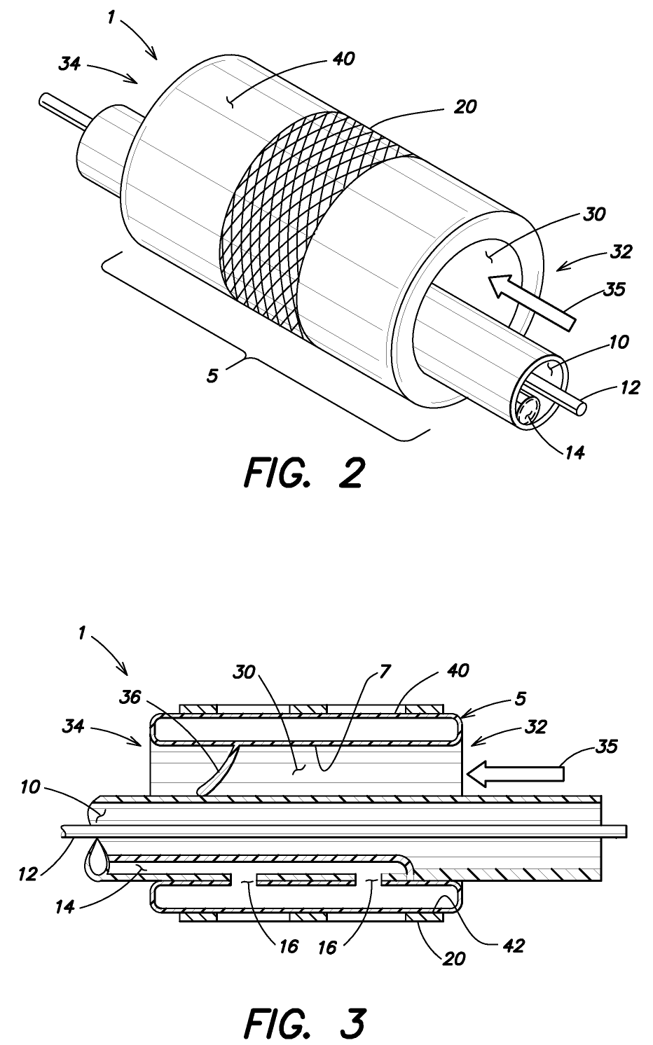

Once at the treatment site, the balloon assembly is inflated. When inflated, as illustrated in FIGS. 2 and 3, the balloon of the balloon 5 expands, and the outer surface 40 of the expanding balloon 5 expands the stent 20 such that the stent 20 engages the body lumen wall at the treatment site, for example, the location of a valve in the body lumen. In this fashion, the stent 20 is implanted into the body lumen at the treatment site, as explained in further detail below.

When the balloon 5 is inflated, the toroidal shape of the balloon 5 creates a body fluid lumen 30 that extends in a longitudinal direction and is open at a distal end 32 and a proximal end 34 of the balloon 5. When inflated, body fluid lumen 30 permits fluid flowing through the lumen being treated to pass, as indicated by arrow 35 in FIGS. 2 and 3, thus disrupting the flow of the body fluid to a lesser extent than if a non-toroidal balloon were to be used. A body fluid lumen valve 36 is located within the body fluid lumen 30 to prevent backflow of body fluid. The body fluid lumen valve 36 can, in certain embodiments, essentially function in place of the natural valve while the implantation of the stent is taking place.

In this embodiment, the balloon 5 is configured such that, when inflated, the outer surface 40 of the balloon 5 extends contiguously from the distal end 32 of the balloon 5 to the proximal end 34 of the balloon 5. This permits the outer surface 40 of the balloon 5 to maximize contact with an inner surface 42 of the stent 20. In this fashion, the expansion forces that the balloon imparts to the stent can be spread evenly along the length of the stent, which can in certain situations permit a more even implantation of the stent in a body vessel.

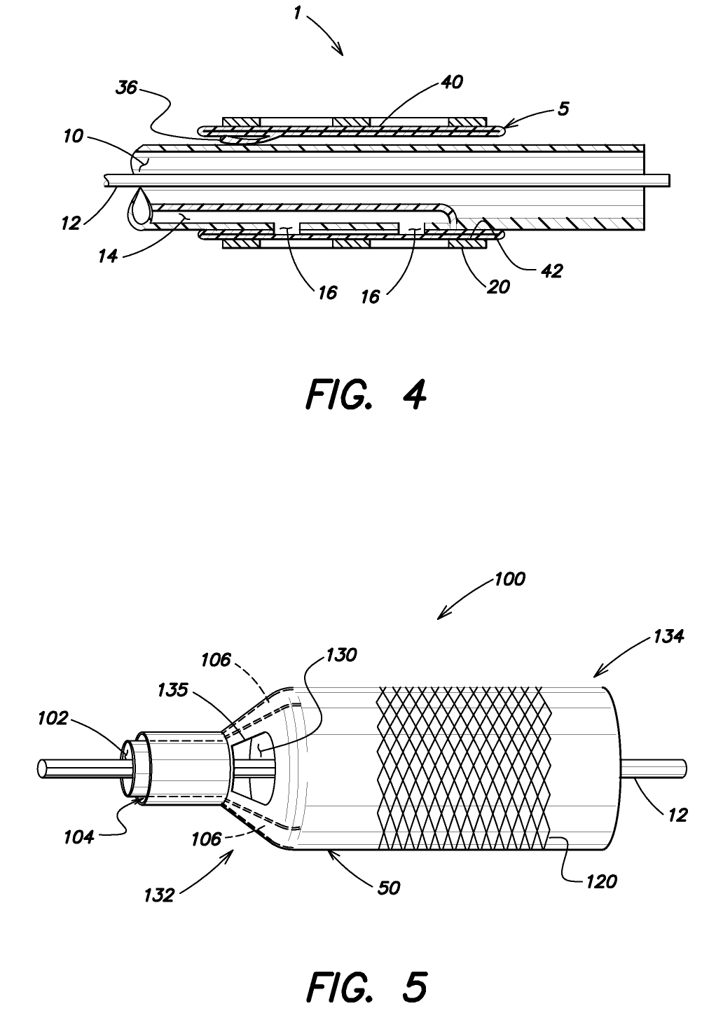

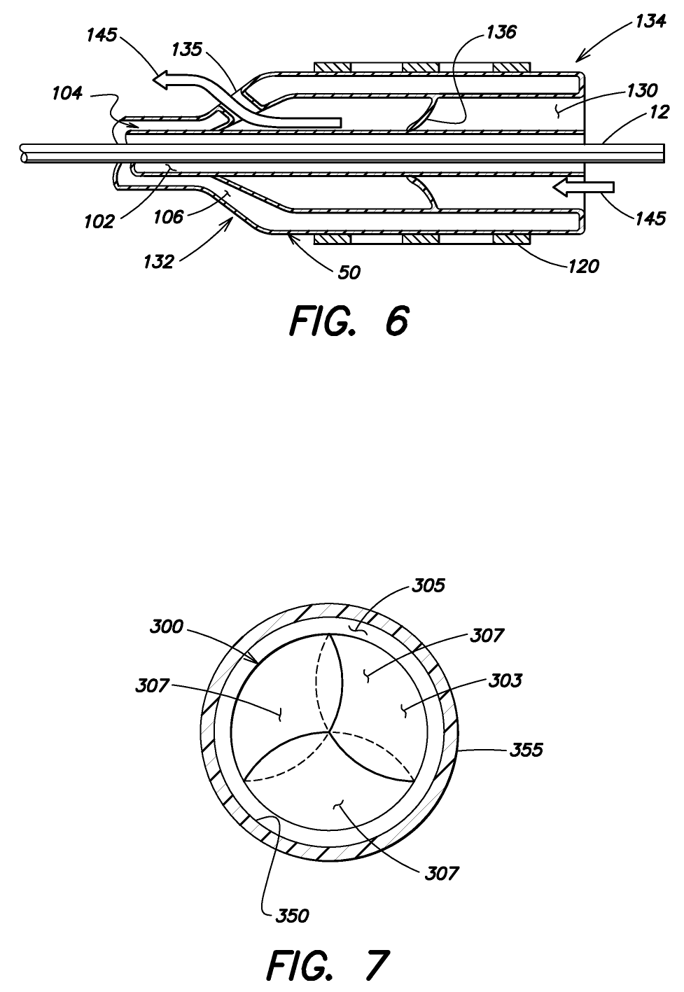

Another embodiment of a balloon catheter is illustrated in FIGS. 5 and 6. In this embodiment, a catheter 100 comprises concentric lumens 102 and 104. The innermost lumen 102 serves as a guide wire lumen, while the outer balloon inflation lumen 104 is in fluid communication with a balloon 50 via channels 106 to permit inflation of the balloon. A stent 120 is crimped around the balloon 50 while the balloon 50 is in an uninflated state, and after being located at a treatment site, the balloon 50 is inflated to expand and implant the stent 120. The balloon 50, once inflated, defines a body fluid lumen 130 that is open at a distal end 134 of the balloon and is open at a proximal end 132 of the balloon by means of windows 135 between the channels. Fluid can flow in through the distal end 134 of the body fluid lumen 130, through the body fluid lumen 130, and exit at the opposite, proximal end 132 of the body fluid lumen 130 through the windows 135, as indicated by arrows 145 (see FIG. 6). A body fluid lumen valve 136 is located inside the body fluid lumen 130. The body fluid lumen valve 136 is configured to permit fluid flow in a physiological direction while substantially preventing fluid flow in the opposite direction when the balloon 50 is inflated. The body fluid lumen valve 136 is typically oriented in the same direction as the native valve and/or a prosthetic valve on the stent being deployed.

The catheter generally may contain any appropriate number of lumens suitable for the intended application. For example, the catheter includes a balloon fluid lumen which is fluidly connected to the balloon to permit the introduction of fluid into the balloon to inflate the balloon. The balloon fluid lumen also serves to permit the evacuating of the fluid from the balloon when it is desired to deflate the balloon, e.g., for removal of the balloon upon completion of the valve placement. Any appropriate fluid, e.g., a liquid or a gas such as, for example, saline or carbon dioxide gas, may be used to inflate the balloon.

The inner walls of the generally toroid balloon can in certain embodiments contain one or more inflatable support elements along the interior wall of the body fluid lumen, which can be inflated to provide support to the interior walls of the body fluid lumen. This can in certain circumstances help prevent the walls of the body fluid lumen from constricting the body fluid lumen, for example, when the balloon is being inflated. These inflatable support elements can be inflated with an inflation medium from a separate support inflation lumen under a relatively high pressure (for example, a higher pressure than the balloon) to inflate rapidly and open up the body fluid lumen before the balloon itself has fully inflated. For example, as illustrated in FIG. 10, a toroid balloon 500 can include multiple support tubules 510 that are attached to the interior walls 502 of the balloon 500 and extend longitudinally along the length of the balloon. The support tubules 510 are fluidly connected to circumferential support tubules 512 that extend circumferentially about the interior walls 502 of the balloon 500. These interconnected support tubules 510 and circumferential support tubules 512 can be supplied with inflation medium from a separate support inflation lumen, for example, by an annular manifold. Any suitable configuration for the inflatable support element(s) can be employed, for example, one or more spiral support tubules, to support the interior walls of the body fluid lumen. In another embodiment, the balloon is segmented and the segments intercommunicate through fluid channels such that inflation can be achieved through a single fluid line while maintaining inner lumen patency. The segments can be tubular circumferentially or interconnecting spheres when inflated.

The balloon can be prepared from a variety of polymeric materials, such as polyethylene, polyolefins, copolymers of olefins, and combinations of any of these. For example, a polyolefin material available from E.I DuPont de Nemours and Co. (Wilmington, Del.), under the trade name Surlyn™ Ionomer, can be used to form the balloon. Combinations of these materials can also be used.

In certain embodiments, the length of the balloon can range from about 10 mm to about 30 mm (e.g., from about 10 mm to about 20 mm). The diameter of the body fluid lumen, when the balloon in its inflated state, is selected to create a suitable size to permit a desirable level of fluid flow through the body fluid lumen. For example, in some embodiments the inner diameter of the body fluid lumen is from about 10 to about 30 mm when the balloon is inflated. The inner diameter of the body fluid lumen generally will depend on the size of the vessel in which it is being located and the amount of fluid flow desired to be maintained during inflation of the balloon.

In some embodiments, in an uninflated state (for example, as illustrated in FIG. 4), the balloon 5 does not significantly increase the overall diameter of the distal end 6 of the catheter 1. Generally, the outer diameter of the balloon, when in an uninflated state, is selected to permit the distal end of the catheter to be threaded through the necessary body lumens to arrive at the treatment site. For example, in some embodiments, the outer diameter of the uninflated balloon can be from about 4 mm to about 7 mm, depending upon the different biological passages of the human (or animal) body in which the catheter will be used. Generally, the catheter is small enough in diameter to be easily maneuvered through the patient's vascular system. The size of the balloon portions may also vary for different procedures and/or patients.

In certain embodiments, when the balloon is inflated, the cross-sectional area of the body fluid lumen is a significant percentage of the cross-sectional area of the treatment site, such as a valvular area of the heart, so as to permit fluid flow through the treatment site comparable to the flow through the site when the catheter is not inserted. For example, in certain embodiments, the cross-sectional area of the balloon is such that the inflated balloon introduces no more than a moderate pressure drop downstream of the balloon.

In certain embodiments, the outer diameter of the inflated balloon can range from about 20 mm to about 35 mm. Generally, the outer diameter of the inflated balloon will depend on the size of the vessel in which it is being located. For example, the outer diameter of the inflated balloon is generally about 20 mm to about 35 mm for use in replacing an aortic valve in an adult human heart, about 20 mm to about 35 mm when replacing a mitral valve in an adult human heart, and about 20 mm to about 35 mm when replacing a tricuspid valve in an adult human heart. Appropriate sizes can be selected depending on the intended use by obtaining measurements of the vessel in which the valve is to be replaced or the prosthesis is to be implanted. For example, generally, non-adult hearts (e.g., infants' or childrens' hearts) will require smaller outer diameters, as will many non-human hearts (e.g., dogs, cats, etc.).

In certain embodiments, the body fluid lumen, when inflated, has an inner open area that is at least about 40% (e.g., at least about 50%, at least about 60%, at least about 70%, at least about 80%, or at least about 90%) of the cross-sectional area of the balloon assembly (which will correspond with the cross-sectional area of the body lumen at the treatment site) when inflated. In certain embodiments, for example, blood (or other fluid) flow through the body fluid lumen of an inflated balloon can be at least about 40% (e.g., at least about 50%, at least about 60%, at least about 70%, at least about 80%, or at least about 90%) of the blood (or other fluid) flow through the body lumen at the treatment site without the balloon in place.

The catheter can be configured to possess desired degrees of pushability, trackability, crossability and torque transmission to the distal catheter end of the catheter as such is applied to the proximal end of the catheter. For example, in certain embodiments the catheter can be configured to have adequate strength for pushability (the ability to transmit force from the proximal end of the catheter to the distal end of the catheter) and resistance to buckling or kinking. In certain embodiments, the catheter can be configured to have adequate flexibility to permit the distal portion of the catheter to track the guide wire through small tortuous vessels or body lumens to reach the area to be treated, for example, by providing at least the distal portion of the catheter with elastomeric properties to improve flexibility. In certain embodiments, the crossability (the ability to navigate the catheter across narrow restrictions or obstructions in the vasculature) can be optimized.

The pushability, trackability, crossability, torque transmission, and other characteristics of the catheter can be adjusted or selected by carefully choosing the catheter material and its physical characteristics, such as wall thickness. For example, because these catheters are frequently inserted for long distances, it is generally desirable to minimize the friction between the guide wire and the surface of the catheter lumen by constructing the catheter from a lubricious material such as a high-density polyethylene (HDPE), polytetrafluoroethylene (PTFE) or other lubricious material(s), either alone or in combination.

In certain embodiments where it is desirable to have different properties or characteristics at different parts of the catheter, the catheter can comprise multiple portions (e.g., two, three, four or more portions) comprising different materials and/or physical characteristics. For example, a tip portion can be provided that is more resilient than the remainder of the catheter lumen for better crossability and to provide a softer leading end of the catheter for abutting internal membranes of the body and the like. Different materials include different polymeric materials from one another, for example, or similar polymers of different densities, fillers, crosslinking or other characteristics. In particular, a portion of a catheter lumen can comprise a material chosen for flexibility to follow a body lumen's path while another portion can comprise a material chosen for axial and/or torque transmission.

The catheter can include any number of internal lumens to provide functionalities to the distal end of the device. Typical lumens included within catheters include a guide wire lumen, a balloon inflation lumen, and/or a support inflation lumen. In some embodiments, the catheter may comprise more than one balloon fluid lumen (e.g., two, three, four or more balloon fluid lumens). These lumens can be used to introduce fluid into different portions of the balloon, for example, to allow for more even inflation of the balloon. In some embodiments, the balloon may comprise multiple balloon sections (e.g., two, three, four or more balloon sections).

In some embodiments, the catheter may further comprise a guide wire lumen through which a guide wire can pass. In some embodiments, the guide wire lumen extends substantially along the full length of the catheter. In other embodiments, the guide wire lumen extends over only a portion of the catheter, e.g., the distal portion of the catheter. In these configurations, the guide wire lumen opens to the exterior of the catheter at a guide wire port, and the guide wire passes through the port and into the guide wire lumen.

As previously noted, the catheter includes at least one body fluid lumen valve disposed in the body fluid lumen that provides the ability to control the direction of fluid flow through the body fluid lumen, as well as the hemodynamics of the fluid flow. More specifically, the body fluid lumen valve allows blood flow at the treatment site in a physiologic direction, while effectively blocking retrograde flow (back flow). As illustrated in FIGS. 3, 4 and 6, the body fluid lumen valve 36 is attached to an inner surface 7 of the balloon 5 and extends across the diameter of the body fluid lumen 30.

The body fluid lumen valve 36 can be formed in any known manner. The body fluid lumen valve is chosen to provide such physiologic characteristics as reducing hemodynamic disturbance from the natural state, and reduced risk of thrombus formation. The body fluid lumen valve can be designed to allow blood flow in a physiologic direction (that is, forward flow of the blood through the biological passage), block back flow (also referred to as retrograde flow) of blood through the device, and collapse sufficiently to allow the catheter to be passed through the vasculature to the treatment site. In some embodiments, the components of the body fluid lumen valve (for example, leaflets) are sufficiently flexible to open and close smoothly, with minimal pressure drop across the body fluid lumen valve and without creating undue turbulence or hemolytically damaging the blood cells.

The body fluid lumen valve can be fabricated to any desired size, depending upon the particular application (for example, heart valve, or other biological passage in the body), and particular patient (for example, a young patient such as a young child, or an elderly patient). Generally, the body fluid lumen valve will be sized to fit the balloon, which will be sized itself in accordance with the particular application and patient.

The body fluid lumen valve can be provided at any position within the body fluid lumen. For example, in some embodiments, positioning the body fluid lumen valve in the proximal or distal end of the body fluid lumen can improve valve function and allow for a smaller collapsed profile. When the body fluid lumen valve is located at a proximal or distal end of the body fluid lumen, the leaflets of the body fluid lumen valve can extend beyond the balloon when the balloon is collapsed, thereby providing improved profile of the collapsed device. The position of the body fluid lumen valve within the body fluid lumen can be such that it is positioned as closely as possible to the native valve's anatomical position, to provide improved valve function during the procedure.

In some embodiments, the body fluid lumen valve may be configured to passively respond to differential pressures on either side of the valve. An active body fluid lumen valve could, in other embodiments, be incorporated into the body fluid lumen, optionally to be controllable from the proximal catheter end. Generally, the body fluid lumen valve comprises an occluder that is moved aside during forward flow of blood through the device, and blocks backflow through the lumen. The body fluid lumen valve is generally suitably durable to withstand pressures within the biological passage to be treated, but flexible enough to move within the device to allow blood or fluid flow through the body fluid lumen.

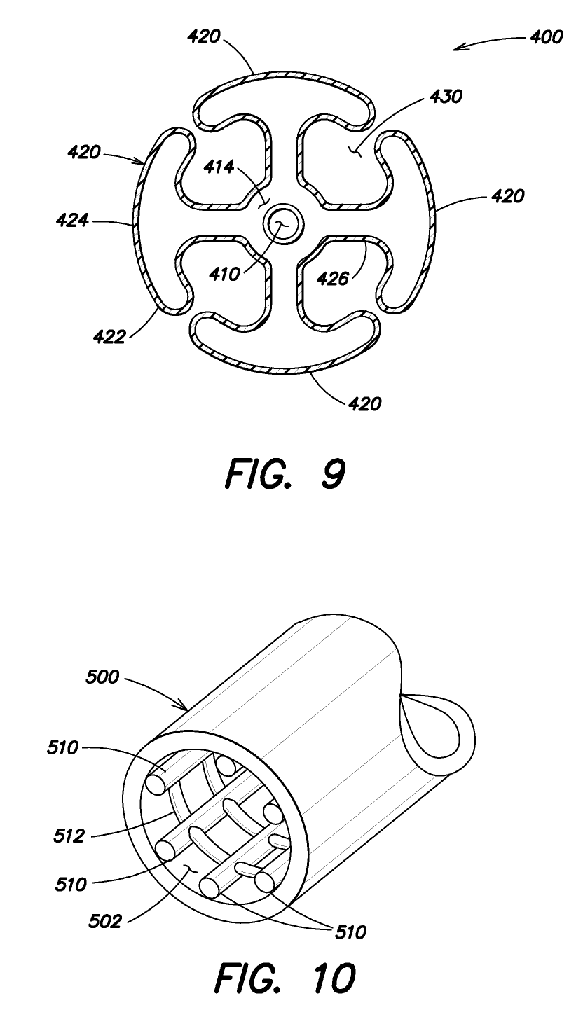

In some embodiments, the balloon can include multiple (e.g., two, three, four or more) body fluid lumens or even a material that permits passage of blood (e.g., a porous material). As one example, as illustrated in FIG. 9, a balloon catheter 400 includes a guide wire lumen 410 and a balloon inflation lumen 414 that is in fluid communication with four balloons 420. Each of the balloons 420 is generally T-shaped in cross-section, with an outer portion 422 of the T having an arced outer surface 424 and an inner portion 426 of the T in fluid communication with both the outer portion 422 and the balloon inflation lumen 414. Upon inflation of the balloons 420, four body fluid lumens 430 are formed, each of which are open at a distal end and a proximal end to a body lumen to permit body fluid found in the body lumen to pass from the distal end to the proximal end (or vice-versa). When inflated, the arced outer surfaces 424 of the balloons 420 form a substantially cylindrical shape of a diameter sufficient to expand a stent (not illustrated) that surrounds the balloons 420 and implant the stent in the body lumen. Each of the body fluid lumens 430 can further include one or more valves configured to permit the flow of fluid in a single direction through the body fluid lumens 430 and prohibit fluid flow in the opposite direction.

As shown in the example of FIG. 7, the body fluid lumen valve may be provided in the form of a flexible leaflet valve 300. The body fluid lumen valve shown in FIG. 7 is a tricuspid valve, and as such, it can be used to mimic the aortic valve. In this embodiment, the flexible leaflet valve 300 comprises a generally arcuate center portion 303 and a peripheral cuff 305. The center portion 303 of the valve 300 comprises three leaflets 307, although it is understood that there could be any desired number of leaflets in the flexible valve, for example, one, two or four leaflets. The peripheral cuff 305 can be used to attach the valve to the inner surface of the balloon, for example, by suturing, biocompatible adhesive, or other suitable attachment methods. Alternatively, the peripheral cuff can be integrally formed in the balloon.

The flexible leaflet valve 300 is disposed within the body fluid lumen 350. The diameter of the arcuate portion 303 can in certain embodiments be substantially the same as the inside diameter of the body fluid lumen 350 when the balloon is inflated. In such embodiments, the peripheral cuff 305 is disposed substantially parallel to the walls of the body fluid lumen 350. Thus, when the balloon 355 is inflated, the valve 300 is expanded and spans the area of the body fluid lumen 350. In some embodiments, when the balloon 355 is in a deflated state, the valve 300 collapses within the balloon so as to substantially conform to the outer dimensions of the collapsed balloon. In certain embodiments, the peripheral cuff 305 is fabricated of a flexible material, to allow the cuff to collapse when the balloon 355 is in an uninflated state. In this way, the valve may not significantly alter the overall diameter of the device.

In some embodiments, each valve leaflet is partially attached to the peripheral cuff along the circumference of the peripheral cuff, with the free end of the leaflet overlapping the adjacent leaflet, such that the leaflets can slide over each other. In this fashion, the leaflets can remain partially overlapping when the anatomy at the treatment site prevents the peripheral cuff from fully expanding, and the leaflets can remain essentially wrinkle-free. By selecting an appropriate leaflet height to diameter ratio, the likelihood of prolapse of the valve can be reduced. For example, a height-to-diameter ratio of about 1 can in some embodiments reduce the likelihood of prolapse.

The material for the leaflets can be a synthetic resin foil for example, a foil of flexible polyurethane. Other materials include silicones, Teflon™, and other polymers. Generally, the majority of the leaflet area consists of a thin membrane. In some embodiments, the area of the leaflets forming the commissural areas can be thicker and/or more rigid, to provide added support for the valve leaflets. In some embodiments, mammalian tissue (such as porcine or bovine pericardium or the like) can be used to form the leaflets. The peripheral cuff 205 can be fabricated of similar materials, and can be formed of a material that is the same as, or different from, the material used to fabricate the leaflets of the valve.

In some embodiments, the leaflets of the valve can be attached to the sheath individually. In these embodiments, no peripheral cuff is included in the device. In these embodiments, the leaflets can be attached to the sheath using sutures, biocompatible adhesive, a combination of the two, or any other suitable attachment mechanism.

Alternatively, the valve can be fabricated to include standardized leaflet structures utilizing some or all of the methodologies described in U.S. Pat. No. 6,945,957, 5,928,281, and 4,888,009.

While the embodiment illustrated in FIGS. 2 and 3 illustrate a catheter having a single balloon thereupon, the catheter can include any number of individual balloons in a number of configurations, depending upon the particular application. For example, a catheter having multiple balloons, each having a separate inflation lumen, can be used to expand and implant a stent in an upstream-to-downstream manner similar to that described above, by inflating the upstream balloon(s) prior to the downstream balloon(s).

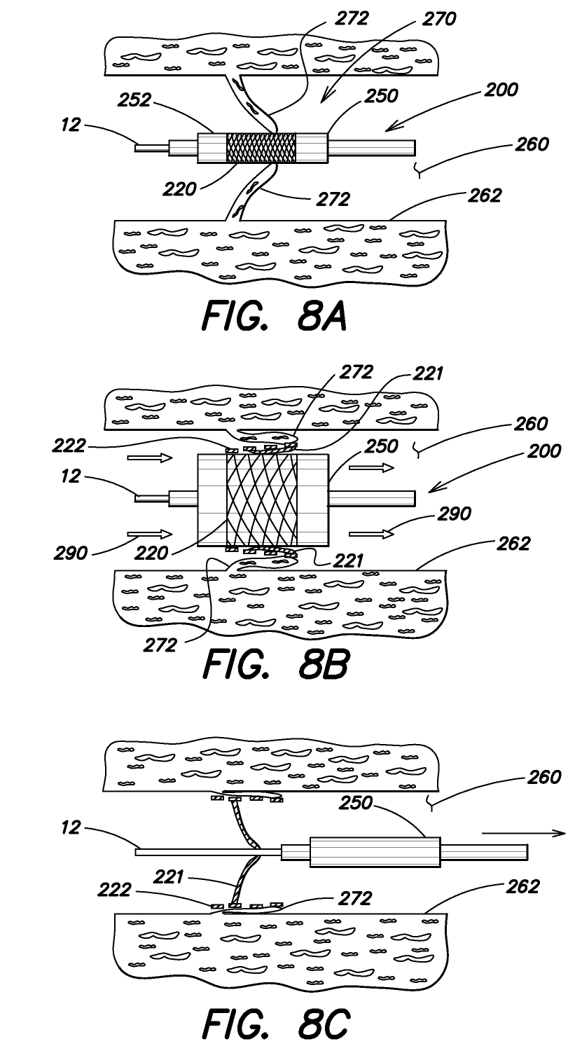

A method of implanting a stent valve is illustrated in FIGS. 8a-8c. A balloon catheter 200 is provided with a stent valve 220 crimped to the outside of a balloon 250. The stent valve 220 includes prosthetic valve leaflets 221, which are initially contained between the stent valve body 222 and an outer surface of the balloon 252. The catheter 200 is inserted into a body lumen 260 and threaded to a treatment site 270, in which a native valve 272 is located (see FIG. 8a). The balloon 250 is positioned such that the stent valve 220, when expanded, will press the native valve 272 against the body lumen walls 262, and the balloon 250 is inflated (see FIG. 8b). The inflation of the balloon 250 expands the stent valve 220, trapping the native valve 272 between the stent valve body 222 and the body lumen walls 262 such that the native valve 272 effectively ceases to impede fluid flow through the body lumen 260. Meanwhile, while the balloon 250 is inflated, fluid is able to pass through a body fluid lumen (not illustrated) in the balloon 250, thus maintaining flow through the body lumen 260 and avoiding pressure buildup upstream of the balloon 250. Fluid flow is indicated by arrows 290. A valve (not illustrated) contained within the body fluid lumen prevents retrograde flow of the body fluid. Once the stent valve 220 has been expanded sufficiently to become implanted in the body lumen 260, the balloon 250 is deflated and the catheter 200 is removed, leaving the stent valve 220 in place (see FIG. 8c). Upon deflation and removal of the balloon 250, the prosthetic valve leaflets 221 of the stent valve 220 unfold and begin to function in place of the native valve 272.

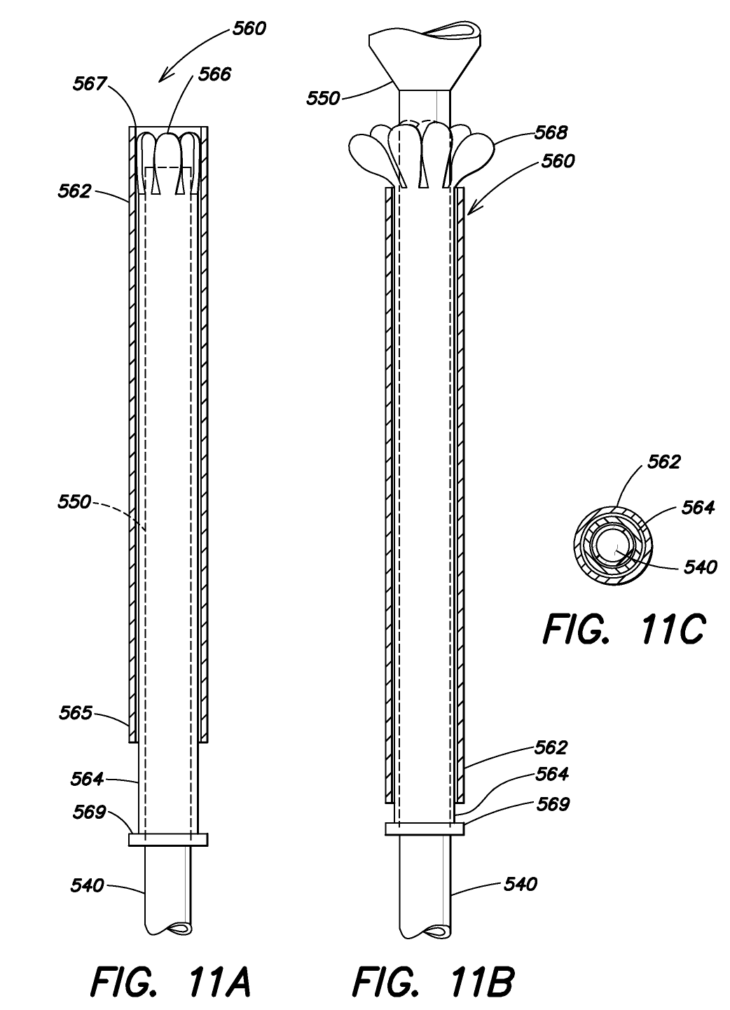

Retrieval of the balloon following deployment of the prosthetic valve can be assisted in some embodiments achieved with a segmented flared extraction tube. As illustrated in FIGS. 11A, 11B and 11C, an extraction tube 560 is used to assist in inserting and extracting a balloon catheter 540. FIG. 11A shows a cross-sectional side view of the extraction tube 560 containing the catheter 504 in a pre-deployment state with the catheter fully contained in the extraction tube, and FIG. 11B shows a cross-sectional side view of the extraction tube with the catheter 540 in a deployed state in which balloon 550 is inflated. FIG. 11C shows a cross-sectional end view of the extraction tube 560. The extraction tube 560 includes an outer tube 562 and an inner tube 564. The extraction tube includes a proximal end 565 and a distal end 567. The inner tube is positioned concentrically within the outer tube and the inner tube has a flared end 566 at the distal end having flared segments 568. The inner tube is slideable within the outer tube for deployment and retraction of the catheter 540 as described below. In at least one embodiment, the inner tube extends out of the outer tube at the proximal end, and the inner tube may include an annular ring 569 that functions as a stop mechanism to limit the distance that the inner tube may slide within the outer tube.

Prior to deployment, the balloon 550 is contained within the extraction tube in its uninflated state, as shown in FIG. 11A. During insertion of the catheter 540, the extraction tube may be inserted through the incision with the catheter 540. In at least one embodiment, the length of the extraction tube may be substantially less than the length of the catheter 540, such that during implantation, the proximal end of the catheter extends out of the proximal end of the extraction tube, with the distal end of the catheter contained within the extraction tube and positioned near the distal end of the extraction tube. After inserting the extraction tube through the incision, the extraction tube may be slid through an artery until its distal end is positioned at approximately the treatment site. The catheter 540, and with it the balloon 550, is slid from within the extraction tube until the balloon is in place at the treatment site, where it can be inflated for use.

In some embodiments, the use of the extraction tube eases the process of withdrawing the balloon and the catheter from the treatment site after use. In preparation for catheter withdrawal, the inner tube may be slid within the outer tube until the annular ring 569 contacts the outer tube to push the flared segments 568 out of the distal end of the outer tube. The inner tube can, for example, be slid approximately 1 cm to expose the flared segments. Extraction of the catheter 540 begins with the deflation of the balloon 550. The catheter 540, with the uninflated balloon 550 thereupon, is then slid within the extraction tube such that the uninflated balloon is pulled through the flared end of the inner tube. The inner tube can function as a funnel, causing the uninflated balloon to collapse as it is drawn into the inner tube. The catheter 540 may be completely drawn into the extraction tube, at which point the extraction tube itself is withdrawn. It is understood, based on the disclosure provided herein, that the device and methods disclosed are applicable to any application where a catheter balloon or similar device is employed to place an implant, including other replacement valve needs, for example, treatment of the esophagus or other biological passages of the body where controlled flow of biological fluids is desired during treatment and venous applications (such as, for example, failure of competence of venous valves).

Embodiments can also be used to implant endoluminal prostheses to a location near a biological valve, where the prosthesis does not itself contain a valve and is not intended to replace the biological valve but the procedure of implanting the prosthesis might at least temporarily block fluid flow and/or affect functioning of the native valve. For example, where a stent is to be located just upstream or just downstream of a biological valve, inflation of a balloon to expand the stent might impinge upon the native valve, for example, by pinning the valve in an open position, for the duration of time that the balloon is inflated. In such a circumstance, the body fluid lumen valve can function in place of the native valve and prevent retrograde fluid flow while the procedure is taking place.

Embodiments can further be used to implant an endoluminal prosthesis in an area of a body lumen not containing a valve, for example in angioplasty, such as coronary angioplasty, peripheral angioplasty, renal angioplasty, and/or carotid angioplasty. An endoluminal prosthesis, for example, a stent, can be deployed in an area where such treatment is desired, for example, at a site of an embolism and/or a stenosis. Using an embodiment of the balloon catheter having a body fluid lumen extending longitudinally through the inflated balloon may prevent the reduction or cessation of fluid flow in the body lumen while the stent is being deployed, which can in certain circumstances prevent pressure build-up upstream of the inflated balloon and/or permit more accurate placement of the stent, as well as preventing disruption of fluid flow downstream of the deployment site. These embodiments may optionally include a valve in the body fluid lumen to reduce or prevent retrograde fluid flow while the balloon is inflated.

What is claimed is:

1. A catheter comprising:
an elongated tubular member having a proximal end and a distal end;
an inflatable balloon assembly disposed at the distal end of the elongated tubular member, the balloon assembly including a balloon having an inflated state and an uninflated state, the balloon comprising a proximal end, a distal end, and an outer surface; and
a balloon inflation lumen in fluid communication with the balloon;
wherein, when the balloon is in the inflated state, the outer surface of the balloon is contiguous from the proximal end of the balloon to the distal end of the balloon, and the lo balloon, when in the inflated state, includes a body fluid lumen that is open at the proximal end of the balloon and at the distal end of the balloon.

2. The catheter of claim 1, the balloon assembly further comprising a body fluid lumen valve moveable between a first position and a second position, wherein the body fluid lumen valve occludes the body fluid lumen to a greater extent in the first position than in the second position.

3. The catheter of claim 2, wherein the body fluid lumen valve substantially prevents a fluid from flowing through the body fluid lumen in a first direction when the body fluid lumen valve is in the first position.

4. The catheter of claim 2, wherein the body fluid lumen valve comprises a leaflet.

5. The catheter of claim 4, wherein the body fluid lumen valve comprises three leaflets.

6. The catheter of claim 1, further comprising a guide wire lumen.

7. The catheter of claim 1, the balloon assembly comprising at least two balloons.

8. The catheter of claim 1, wherein the balloon inflation lumen is in fluid communication with the balloon via at least one opening.

9. The catheter of claim 1, wherein, when the balloon is inflated, the body fluid lumen has a cross-sectional area that is at least about 40% of the cross-sectional area of the balloon assembly when the balloon is inflated.

10. The catheter of claim 1, wherein, when the balloon is inflated, the body fluid lumen has a cross-sectional area that introduces no more than a moderate pressure drop downstream of the balloon when the balloon is inflated.

11. A medical device, comprising:
an inflatable balloon assembly, including a balloon having an inflated state and an uninflated state, the balloon comprising a proximal end, a distal end, and an outer surface; and
an endoluminal prosthesis surrounding the balloon assembly and adjacent the outer surface of the balloon;
wherein the balloon, when in the inflated state, includes a body fluid lumen that is open at the proximal end of the balloon and at the distal end of the balloon.

12. The medical device of claim 11, the balloon assembly further comprising a body fluid lumen valve moveable between a first position and a second position, wherein the body fluid lumen valve occludes the body fluid lumen to a greater extent in the first position than in the second position.

13. The medical device of claim 12, wherein the body fluid lumen valve substantially prevents a fluid from flowing through the body fluid lumen in a first direction.

14. The medical device of claim 12, wherein the body fluid lumen valve comprises a leaflet.

15. The medical device of claim 12, wherein, when the balloon is in the inflated state, the outer surface of the balloon is contiguous from the proximal end of the balloon to the distal end of the balloon.

16. The medical device of claim 11, the balloon comprising an interior surface that defines the body fluid lumen, the medical device further comprising an inflatable support element that extends along the interior surface of the balloon.

17. The medical device of claim 16, wherein the inflatable support element comprises a spiral tubule in fluid communication with a support inflation lumen.

18. The medical device of claim 16, wherein the inflatable support element comprises at least two tubules in fluid communication with a support inflation lumen.

19. The medical device of claim 11, wherein, when the balloon is inflated, the body fluid lumen has a cross-sectional area that is at least about 40% of the cross-sectional area of the balloon assembly when the balloon is inflated.

20. The medical device of claim 11, wherein the endoluminal prosthesis comprises a prosthetic valve.

21. The medical device of claim 11, wherein the endoluminal prosthesis comprises a prosthetic aortic valve.

22. The medical device of claim 11, wherein the endoluminal prosthesis comprises a prosthetic mitral valve

23. An endoluminal prosthesis implant device for a body vessel containing a flowing fluid, comprising:
an inflatable balloon assembly, including a balloon having an inflated state and an uninflated state, the balloon comprising a first end, a second end, and an outer surface; and
an endoluminal prosthesis surrounding the balloon assembly and adjacent the outer surface of the balloon;
wherein the balloon assembly is configured to permit fluid to pass from the first end of the balloon to the second end of the balloon when the balloon is in the inflated state.

24. The endoluminal prosthesis implant device of claim 23, wherein the balloon assembly is further configured to prevent a fluid to pass from the second end of the balloon to the first end of the balloon when the balloon is in the inflated state.

25. The endoluminal prosthesis implant device of claim 23, wherein the balloon assembly comprises a valve that prevents a fluid to pass from the second end of the balloon to the first end of the balloon when the balloon is in the inflated state.

26. The endoluminal prosthesis implant device of claim 23, wherein, when the balloon is in the inflated state, the outer surface of the balloon is contiguous from the proximal end of the balloon to the distal end of the balloon.

27. The endoluminal prosthesis implant device of claim 23, wherein the endoluminal prosthesis comprises a prosthetic valve.
